(19) United States
(12) Patent Application Publication
(10) Pub. No.: US 2010/0307210 A1
(43) Pub. Date: Dec. 9, 2010

(54) Vertical Manure Converter and Process including Activated Carbon in an Organic Mixture

(76) Inventors: Frank Rosenbaum, Oro Valley, AZ (US); James Sheppard, Midland, MI (US); Kurt Keiffer, Carson City, MI (US); Jason Simon, Fowler, MI (US); Michael McCrackin, Carson City, MI (US)

Correspondence Address: MILLER, CANFIELD, PADDOCK AND STONE, 277 SOUTH ROSE STREET, SUITE 5000, KALAMAZOO, MI, 49007, US

(21) Appl. No.: 12/740,604

(22) PCT Filed: Nov. 25, 2008
(86) PCT No.: PCT/US08/84690

§ 371 Date: Apr. 29, 2010

Related U.S. Application Data
(60) Provisional application No. 60/990,143, filed on Nov. 26, 2007.
(60) Provisional application No. 61/046,131, filed on Apr. 18, 2008.
(60) Provisional application No. 61/049,604, filed on May 1, 2008.

(51) Int. Cl. C05F 3/00; C12M 1/00; C12M 1/36

(52) U.S. Cl. 71/21; 435/290.4; 435/286.7

(57) ABSTRACT

A vertical manure converter and related process of using activated carbon in an activated carbon/organic material mixture to process organic waste. Inducing air, preferably compressed, into a chamber of the converter in combination with reusing activated carbon can result in a continuous process. The temperature can be controlled by regulating the air into the chamber. The vertical manure converter takes organic waste and uses heat to accelerate the composting process wherein final products can be purified water and ash fertilizer.

The disclosure claims partial priority to U.S. Provisional Patent Application Ser. No. 60/990,143, filed Nov. 26, 2007, entitled “Manure Processing System for Wastewater Treatment and Fertilizer Extraction;” U.S. Provisional Patent Application Ser. No. 61/046,131, filed Apr. 18, 2008, entitled “Process including Injecting Compressed Air into an Activated Carbon/Organic Mixture;” and U.S. Provisional Patent Application Ser. No. 61/049,604, filed May1, 2008, entitled “Vertical Cylinder Manure Converter.”

### BACKGROUND

This disclosure relates to converters and processes of making fertilizer and treating waste by-products. More specifically, the processes include extracting concentrated fertilizer nutrients from animal manure, urine and wastewater and cleaning wastewater. A vertical manure converter takes organic wastes and uses heat to accelerate the composting process wherein a final product can be easily handled, transported and used as valuable fertilizer product.

Animal feeding operations (AFO) are constrained by animal manure, animal urine and water contaminated by animal manure, urine and other nutrients that water comes into contact with (wastewater). AFOs must continually dispose of manure and wastewater, which is a difficult, costly and dangerous process due to the presence of methane gas. Wastewater disposition is especially difficult since wastewater nutrients generally exceed state and Federal clean water standards. Evaporation is too slow of a process for AFOs.

U.S. Pat. Nos. 6,982,068 and 7,199,069, which are incorporated by reference, disclose a method for oxidizing organic compounds in a controlled manner within a bed of activated carbon. The bed of activated carbon is exposed to a source of molecular oxygen, such as air, and is controlled within a temperature range whereby the molecular oxygen is slowly oxidizing the activated carbon. Under this controlled set of conditions, the activated carbon may oxidize organic compounds present within the bed of activated carbon.

The disclosure includes flowing a gas including a source of oxygen molecules through the activated carbon bed and heating the bed to an operating temperature range whereby the oxygen molecules are oxidizing the activated carbon. Activated carbon held within the temperature range of 150 degrees C. (302 F) to 375 degrees C. (707 F) and provided with a source of gaseous oxygen, such as air, can be utilized for useful purposes such as the controlled oxidation of oxidizable organic vapors, the controlled oxidation of oxidizable organic liquids, and regeneration of activated carbon containing adsorbed oxidizable organic compounds.

The supply of oxygen provided to a reacting bed in a related method called for flowing air over the top of a bed of a mixture including activated carbon or the presence of air within a bed of activated carbon. In such a method, the pilot was difficult to ignite, extreme continual external heat was required and the process would not properly sustain itself. As such, improvements regarding airflow are advantageous.

The process and apparatus in U.S. Pat. Nos. 6,982,068 and 7,199,069 can handle one batch of product at a time, but they are not designed for continuous flow of materials. Neither a rotary kiln nor a vertical manure separator will operate on a sustained basis with wet manure without improving the process outlined in these patents. An improved vertical cylinder device and process are desirable.

### DEFINITIONS

In this disclosure, an AFO is defined as an animal feeding operation which stables, confines or concentrates animals. AFOs affected by manure and wastewater disposition issues are primarily, but not limited to, the following agricultural activities:
Dairy farms
Swine Farms
Veal/Beef cattle feeding operations
Turkey farms
Chicken (broiler) farms
Chicken (laying) farms
Sheep or lamb farms
Horse farms

In addition to the aforementioned, any process, where animal manure, urine and/or wastewater are a by-product, is included in this disclosure.

Manure is defined as animal excrement generated by the animal's intestinal system and includes bedding, compost and raw materials or other materials comingled with animal excrement or set aside for disposal. Urine is defined as liquid animal excrement generated by the animal's kidney system. Wastewater is defined as water contaminated by contact with manure, urine and other nutrients, such as during the AFO process.

In addition to animal manure and urine, the process is applicable, and therefore expanded, to include any organic matter whether or not requiring waste disposal techniques.

### SUMMARY

The present disclosure provides converters and processes used to convert manure and wastewater to fertilizer and water that meets Federal and state clean water standards. Waste water is heated by various means resulting in concentrated fertilizer and steam or distilled water. Solid organic materials can be separated and allowed to compost or otherwise be processed.

In a process of oxidizing organic compounds using activated carbon, injecting compressed air into an activated carbon mixture with organic materials has numerous potential benefits to improve the process. Injecting compressed air improves airflow and may help ignite the mixture, help sustain the process, require less external heat, or allow the process to work at a temperature range of 300-450 degrees C.

A vertical manure converter to accelerate processing of organic material using an activated carbon/organic material mixture includes a chamber with sections for controlling descent, such as by gravity with panels in the chamber, of the activated carbon/organic material mixture. Air injectors along the chamber induce air into the chamber, such as compressed air in multiple locations and levels. The temperature can be regulating with controlled airflow into the chamber. Several means are disclosed for reusing activated carbon for a continuous process.

A vertical manure converter can incorporate lifting activated carbon from the bottom of the unit, preferably a cylinder, to the top or otherwise reusing the activated carbon. Additional improvements may include: compressed air injector nozzles located throughout the unit; lifting of the activated carbon from the bottom to the top of the unit with an auger or pump; the activated carbon slinger at the top of the unit; the dispersal cones at the top and bottom of the unit; the various flights that slow descent of the activated carbon/manure mixture; the sloping screen to separate activated carbon from ash and sand; the sloping floor at the bottom of the unit to collect ash and sand; a computer system to control the amount of manure and air injected into the manure converter; and hot activated carbon can remain inside the system resulting in a significant increase in throughput of manure. These improvements allow for a continuous flow of organic materials, such as processing wet manure, on a sustained basis. These improvements make an activated carbon process more commercially viable.

### BRIEF DESCRIPTION OF THE DRAWINGS

The above mentioned and other features of this disclosure and the manner of obtaining them will become more apparent, and the disclosure itself will be best understood by reference to the following description of processes taken in conjunction with the accompanying figures, which are given as non-limiting examples only, in which:

FIG. 1 is a flow chart for a boiling process for extracting fertilizer from manure and purifying wastewater;

FIG. 2 is a flow chart for a direct flame burning process for extracting fertilizer from manure and purifying wastewater;

FIG. 3 is a flow chart for, an activated carbon process for extracting fertilizer from manure and purifying wastewater;

FIG. 4 shows a schematic of a vertical cylinder manure converter;

FIG. 5 is a detailed and partially cut away diagram of the complete system using activated carbon for extracting fertilizer from manure and purifying water; and

FIG. 6 shows a perspective view of the vertical manure converter and the mixing reservoir.

The exemplifications set out herein illustrate embodiments of the disclosure that are not to be construed as limiting the scope of the disclosure in any manner. Additional features of the present disclosure will become apparent to those skilled in the art upon consideration of the following detailed description of illustrative embodiments exemplifying the best mode of carrying out the disclosure as presently perceived.

### DETAILED DESCRIPTION

While the present disclosure may be susceptible to embodiment in different forms, the figures show, and herein described in detail, embodiments with the understanding that the present descriptions are to be considered exemplifications of the principles of the disclosure and are not intended to be exhaustive or to limit the disclosure to the details of construction and the arrangements of components set forth in the following description or illustrated in the figures.

The disclosed processes allow for extraction of concentrated fertilizer nutrients from animal manure, urine and wastewater and cleaning wastewater to state and Federal clean water standards. In the first two processes, the potential adverse presence of methane gas can be used as a source of heat.

Boiling Process for Extracting Fertilizer from Manure and Purifying Wastewater

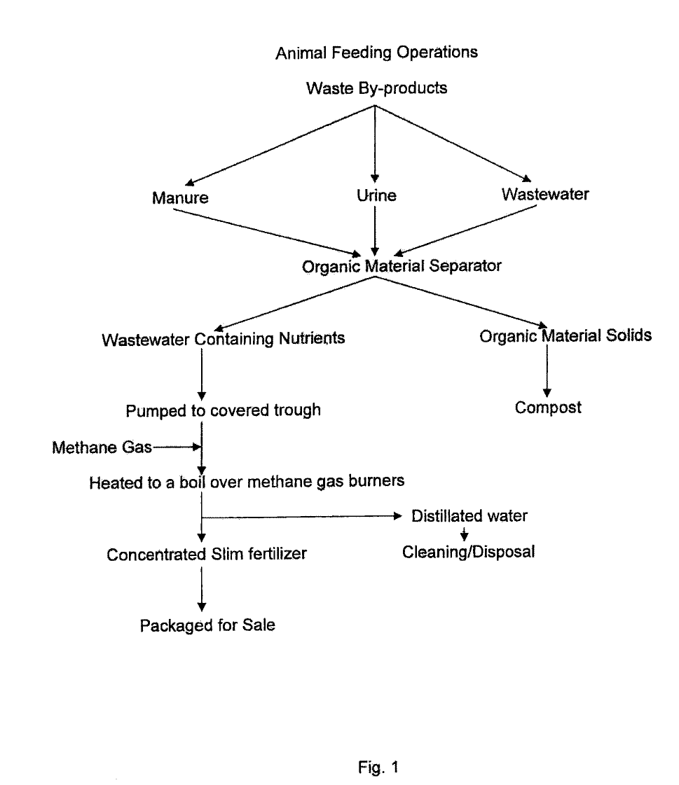

Per FIG. 1, raw manure and/or other waste by-products are separated into organic material solids and wastewater by an organic material separator, such as a mechanical separator.

Organic material can be stacked in a compost pile and allowed to compost at temperatures more than 160 degrees Fahrenheit or otherwise processed. Composted materials are available for animal bedding or returned to fields.

The wastewater stream is directed over a heated, hooded or covered trough. As the wastewater moves through the trough, it is heated to its boiling point. Methane gas, from an anaerobic digester, can be used as a source of energy to produce heat. The methane gas is routed to a burner under the trough where it is ignited to a temperature that boils the wastewater. As wastewater boils, water evaporates into steam which is collected inside the hood and allowed to runoff and be captured as distillated water. The residual wastewater becomes a concentrated slime material rich in nutrients for use as concentrated fertilizer.

Direct Flame Burning Process for Extracting Fertilizer from Manure and Purifying Wastewater

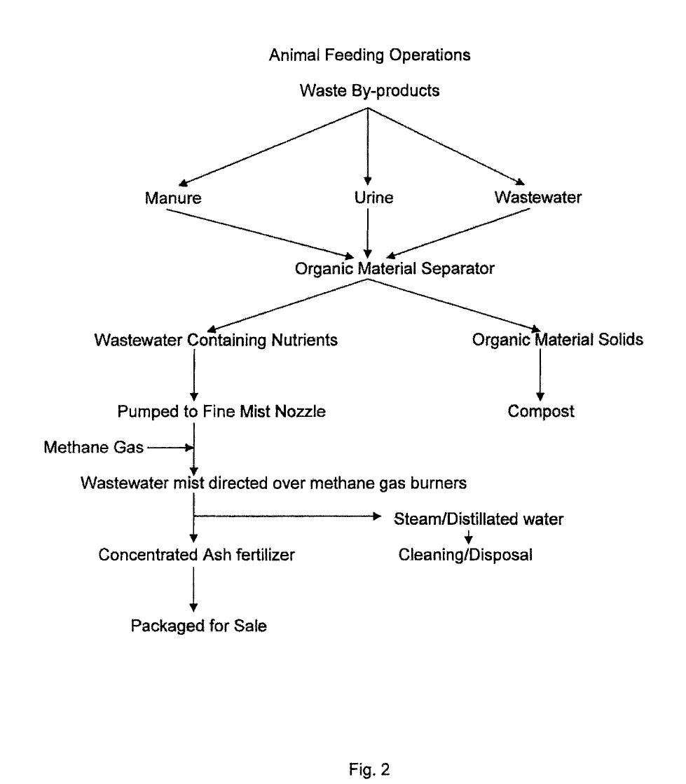

Per FIG. 2, raw manure and/or other waste by-products are separated into organic material and wastewater by an organic material separator, such as a mechanical separator.

Organic material can be stacked in a compost pile and allowed to compost at temperatures more than 160 degrees Fahrenheit or otherwise processed. Composted materials are available for animal bedding or returned to fields.

The wastewater stream is pressurized and directed through a nozzle that converts the wastewater into a fine mist spray. The spray is directed through a direct flame, which incinerates the wastewater resulting in a fine ash fertilizer material, which can be collected in a bin located at the bottom of the incinerator. Methane gas, from an anaerobic digester, can be used as a source of energy to produce the direct flame for the process. The methane gas is routed to a burner inside the incinerator. As wastewater spray flows through the direct flame, water evaporates into steam, which is collected inside the incinerator and captured as distillated water. The bin containing the fertilizer ash is emptied into storage containers for future use.

Activated Carbon Process for Extracting Fertilizer from Manure and Purifying Wastewater

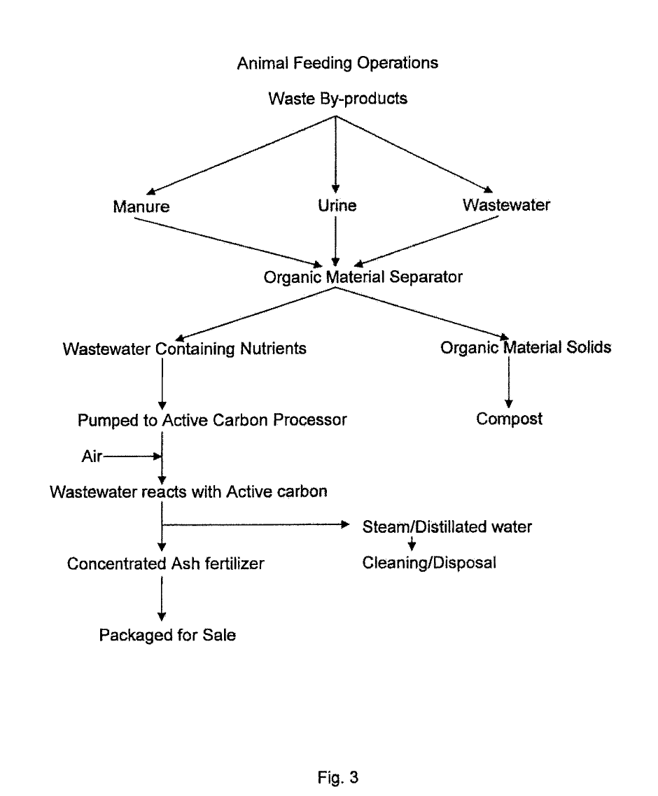

Per FIG. 3, raw manure and/or other waste by-products are separated into organic material and wastewater by an organic material separator, such as a mechanical separator.

Organic material can be stacked in a compost pile and allowed to compost at temperatures more than 160 degrees Fahrenheit or otherwise processed. Composted materials are available for animal bedding or returned to fields.

The wastewater stream and air are directed into a mixer containing activated carbon. The reaction of activated carbon, air and wastewater causes the contents of the mixer to heat to a temperature more than 800 degrees Fahrenheit. After reaching this temperature, the contents of the mixer are separated into ash fertilizer and steam. The steam can be used for heating and cleaning purposes. The ash is removed from the mixer and stored in containers. During the mixing process, phosphorous precipitates on to the activated carbon material. When saturated, phosphorous is removed from active carbon during the active carbon cleaning process. The residual phosphorous is collected in containers and used as fertilizer.

The use of activated carbon can speed the process resulting in concentrated fertilizer and steam/distilled water.

Injecting Compressed Air into an Activated Carbon/Organic Material Mixture

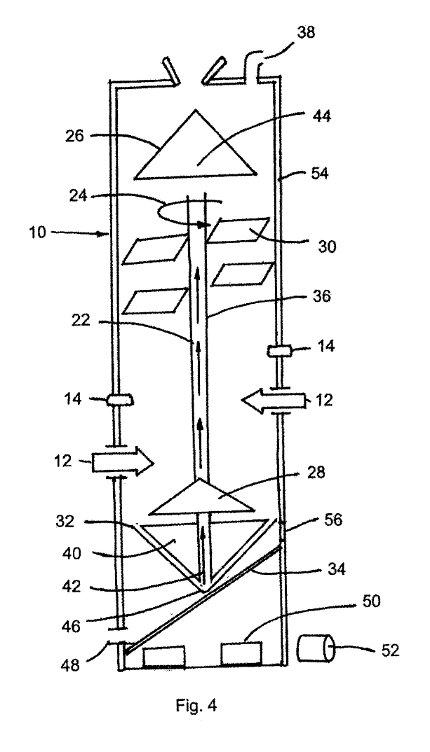

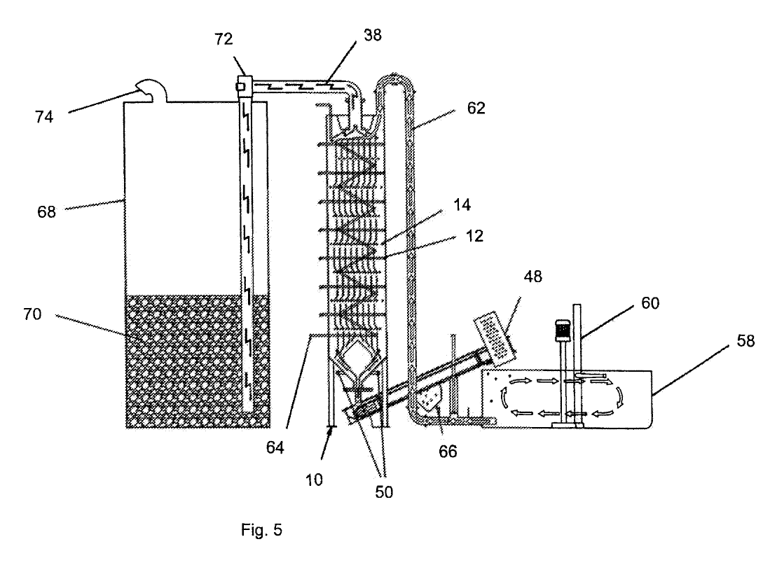

Per FIGS. 4 and 5, a simplified summary of a process includes heating a pre-measured activated carbon mixture with organic materials (such as manure), burning the mixture resulting in ash and distilled water, and then reusing the activated carbon. The method of inducing compressed air into the process can result in significant improvements and allow the process to properly operate. This process is shown as a vertical manure converter (VMC) system 10.

In a process of oxidizing organic compounds using activated carbon, injecting compressed air into an activated carbon mixture with organic materials may improve airflow, help ignite the mixture, help sustain the process, require less external heat, or allow the process to work at a temperature range of 300-450 degrees C. The operating temperature range was increased from 375 degrees C. in the initial disclosure because higher temperatures of 375-450 degrees C. were better suited to sustain a commercial process. This higher temperature exceeds the ignition point of activated carbon.

A pipe or similar air injecting device 12 with spigots or nozzles can pump or inject compressed air onto or into the activated carbon mixture with organic materials. At the initiation of the process, injected compressed air can also help to initially ignite the activated carbon mixture with organic materials. Compressed air could be blown on the coals and injected into the activated carbon mixture with organic materials to sustain the process.

Compressed air can be ambient air, but it may include pure oxygen or variations of nitrogen and oxygen from “air.”

For example, with wet manure, having air flow along the top of an activated carbon mixture with organic materials might get too hot and the process suffocates. A process without proper airflow might require external heat or could get so hot that air would not get to the activated carbon and the process could not sustain itself.

Injecting compressed air, preferably computer controlled, onto or into the activated carbon mixture with organic materials can operate at a range of 300-450 degrees C., such as at 400 degrees C. Various temperature probes 14 throughout the vertical manure converter chamber 16 can relay temperature conditions within the vertical manure converter system 10 to a computer monitor or system 18. The computer monitor, using a series of check valves 20, can increase or decrease the amount of airflow to the vertical manure converter system 10, such as into the chamber 16, to maintain proper operating temperatures. System operations at this relatively low temperature allow for less restrictive equipment and potential uses for waterless commodes, waste treatment, and agricultural uses. It is contemplated that a device twenty feet tall by four square feet could process 250,000 gallons of manure per day resulting in safe water and ash that could be used for fertilizer.

In addition, the vertical manure converter system 10 generates heat that can be captured and used for hot water heat or as steam to drive electric generating equipment. Piping that is part of a closed loop water system can be circulated through the vertical manure converter system 10. Water in the pipes is heated and exits the vertical manure converter system 10 as steam where it is directed to be used either as a source of heat or to drive electrical generating equipment.

### Vertical Manure Converter

A vertical manure converter 10 can incorporate lifting activated carbon from the bottom of the chamber 16, preferably a cylinder, to the top. Additional improvements preferably include: compressed air injector nozzles 12 located throughout the chamber 16; lifting of the activated carbon from the bottom to the top of the chamber 16 with an auger; the activated carbon slinger 24 at the top of the chamber 16; the dispersal cones 26 and 28 at the top and bottom of the chamber 16; the various flights 30 that slow descent of the activated carbon/manure mixture; the sloping screen 32 to separate activated carbon from ash and sand; the sloping floor 34 at the bottom of the unit to collect ash and sand; a computer system 18 to control the amount of manure and air injected into the chamber 16; and hot activated carbon remains preferably inside the vertical manure converter system 10 at all times resulting in a significant increase in throughput of manure. These improvements allow for a continuous flow of organic materials, such as processing wet manure, on a sustained basis.

As depicted in FIG. 4, the operational flow and features of the Vertical Cylinder Manure Converter (VCMC), a type of vertical manure converter system 10 with internal reuse of activated carbon, can be as follows:
Step 1. Manure is pumped to the top of the VCMC where it enters the chamber 16, such as a cylinder.
Step 2. The manure free falls onto the stationary dispersal cone 26, which has rivulets to evenly disperse manure within the chamber 16. The stationary cone 26 also protects an auger pipe 36 area from direct contact with the manure stream.
Step 3. Hot activated carbon is transported to the top of the chamber 16 by an auger 22 located in the center of the chamber 16.
Step 4. The hot activated carbon is slung from the auger 22 (which may spin) at the top of the chamber 16 where the activated carbon comes into contact with the free falling manure.
Step 5. The free falling manure/activated carbon mixture comes into contact with sections 30, like flights (such as angled steel partitions) whose purpose is to slow the descent of the falling manure/activated carbon mixture and provide a means to continually mix and aerate the manure/activated carbon.
Step 6. Computer controlled compressed air is injected into the chamber 16 at selected points to provide adequate oxygen for the activated carbon to react with the manure.
Step 7. Water vapor (steam) is removed from the VCMC at the top of the chamber 16 through a steam exhaust area 38. The steam may then used for heating the incoming manure stream or other purposes.
Step 8. The manure/activated carbon mixture then settles in the chamber 16 where the final reaction of the manure with the activated carbon takes place. This bottom area 40 of the chamber 16 will approach temperatures of 400 degrees C., which completes the conversion of the manure to ash and water vapor.
Step 9. At the bottom area 40 of the chamber 16, activated carbon, ash and sand flow over a gravity screen 32 (such as a sloping grate) that separates sand and ash from the activated carbon.
Step 10. The activated carbon remains on top of the gravity screen 32 and flows into openings 42 in the auger support pipe 36.
Step 11. The auger 22 located inside the auger support pipe 36 transports the hot activated carbon to the top 44 of the chamber 16 to begin the process of mixing with entering manure.
Step 12. Ash and sand fall from the separation screen 32 onto a sloped floor 34 inside the chamber 16. The sloped floor 34 gravity-feeds the ash and carbon to the discharge tube 46. Air pressure from inside the chamber 16 facilitates the feeding of ash and sand to the discharge tube 46.
Step 13. Ash and sand recovered from the vertical manure converter 10 are separated using material separator 48, such as a mechanical separator.
Step 14. Heaters 50, such as propane burners, are located under the sloped flooring 34 of the chamber 16 and are used during the startup process to heat the activated carbon.
Step 15. A mechanical means for drawing in and moving a substance 52, such as a pump or auger drive motor, is housed outside the chamber 16 for maintenance and to keep it away from the heat generated by the vertical manure converter system 10.
Step 16. The chamber 16 can be shrouded in insulation 54, such as fireproof insulation jacket, in order to retain heat within the chamber 16.
Step 17. Temperature probes 14 are located at critical locations within the chamber 16.
Step 18. Temperatures will be monitored throughout the chamber 16 by a computer system 18. The computer system 18 will regulate temperatures within the chamber 16 by increasing or decreasing compressed air. Should temperatures exceed a critical value, the volume of compressed air at the nearest air fixture(s) 12 to the over-temperature area is reduced in order to lower temperatures. Additional compressed air volume can be injected into the chamber 16 to raise temperatures.
Step 19. Various hatches 56 can be available in the unit to remove the auger assembly and for ease of cleaning and maintenance.

FIG. 5 shows a diagram of a complete vertical manure converter system 10 that uses activated carbon for extracting fertilizer from manure and purifying water with arrows showing flow. A separate mixing reservoir 58 can be a bin with a mixing pump 60 for mixing organic materials, such as manure, with activated or reactivated carbon. Unlike the VCMC, the manure/carbon mix is not necessarily made inside the chamber 16. A manure/carbon mix can be moved (i.e. via pump 52) from the mixing reservoir 58, such as through a conduit or pipe 62, to the top of the chamber 16. The manure carbon mixture is processed, such as via a controlled descent, through the chamber 16 of the vertical manure converter, which includes a dispersal cone 28 and a heater 50, such as four non-contact 100,000 BTU propane fired burners, toward the bottom 40 of the chamber 16. The chamber 16 of the vertical manure converter may also include multiple air injectors 12, such as injected compressed air, at various levels of the chamber 16, water lets 64 for steam toward the end of the air stream, a steam supply for a generator and an outlet 38 for an exhaust stream as shown on the top of the chamber 16. Preferably above the mixing reservoir 58, a converter/separator 48 separates sand/ash for collection by a sand/ash collector system 66 and allows raw recycled and reactivated carbon to enter the mixing reservoir 58 for subsequent use in a continuing process.

Via the exhaust area 38, exhaust stream from the chamber 16 may move forward into a container 68 with activated carbon 70, such as in a bed, to filter the exhaust stream. For example, as shown in FIG. 5, a 15,000 gallon container 68 could be partially filled with activated carbon 70 through which the entire exhaust stream passes to minimize remaining odorous emissions. The exhaust stream can be released at the bottom of the activated carbon bed 70. An exhaust blower 72 may be mounted on top of the container 68 to help advance the exhaust stream into the activated carbon bed 70 to be filtered. A filtered exhaust port 74 above the container 68 (after the exhaust stream has passed through the activated carbon bed 70) further ensures clean final exhaust. The carbon collection unit associated with the container 68 can minimize adverse emissions.

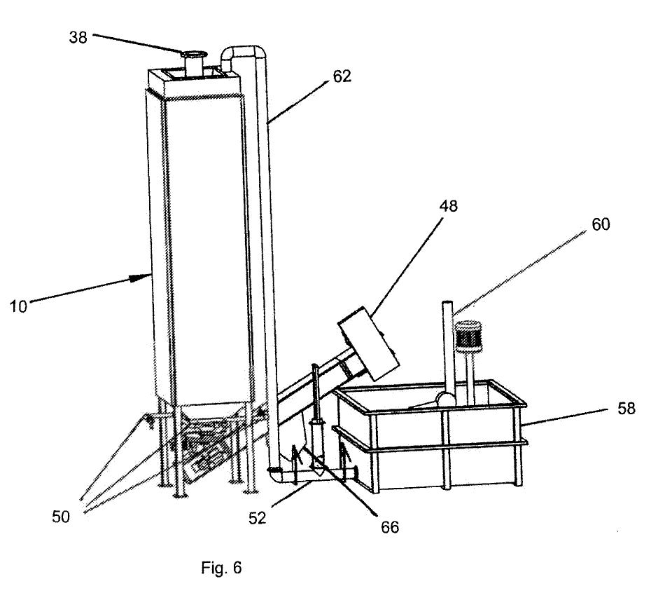

Per FIG. 6, a vertical manure converter system 10 can accelerate processing of organic material using an activated carbon/organic material mixture. The chamber 16 and the mixing reservoir 58 are placed adjacent to each other with the converter/separator 48 above the mixing reservoir 59. The vertical manure converter system 10 includes a chamber 16 with internal sections 30 for controlling descent, such as by gravity with panels in the chamber, of the activated carbon/organic material mixture. One or more dispersal cones 28 can disperse activated carbon/organic material mixture within the chamber 16.

Air injectors 12 along the chamber 16 induce air into the chamber 16, such as compressed air in multiple locations and levels on the chamber 16. The aeration of the descending activated carbon/organic waste helps maintain the proper heat in a continuing process. A computer system 18 can monitor and regulate descent, air flow and temperature. The temperature can be regulating with controlled airflow into the chamber 16. The computer system 16 and temperature probes 14 with relays to the computer system 16 can control the amount of air injected into the chamber 16 via the air injectors 12. In vertical manure converter system 10, an operating temperature is in a range of 300-450 degrees C., more specifically 375-450 degrees C. been useful for a sustainable continuous process.

The mixing reservoir 58 can assist with reusing activated carbon for a continuous process. Recycled or reactivated carbon can be mixed with newly added organic material, which can be computer controlled.

A heater 50 can be associated with the bottom area 40 of the chamber 16, which may be used at various stages of the process including start-up or continuing through a continuous process.

The vertical cylinder converter system 10 and process are not limited to manure. They can be utilized for any organic material that requires accelerated decomposition, and they can also be used for re-generating spent activated carbon on a high volume basis.

A method of treating organic waste may include mixing activated carbon into organic waste to form an activated carbon/organic waste mixture, which can be done internally in the chamber 16 per the vertical cylinder manure converter or partially externally with a mixing reservoir 58.

Next, the descent of the activated carbon/organic waste mixture in a vertical chamber 16 is controlled. The activated carbon/organic waste mixture can be dispersed for better aeration, such as by a top dispersal cone 26. Aeration of the activated carbon/organic waste mixture in the vertical chamber 16 is ideal when the activated carbon/organic waste is dispersed and aerated at several levels within the chamber 16.

With the aeration and temperature control, reusing activated carbon can result in a continuous process of treating organic waste. Reusing activated carbon has several methods including internal with the auger 22 and with a mixing reservoir 58 as detailed above.

The addition of heat accelerates the drying process by rapidly evaporating moisture, including as steam or making purified water. The internal aspect of the activated carbon mixed with organic waste minimizes organic emissions and odor using the internal auger 22. The organic waste is broken down into an ash like substance that can be separated as detailed above. The final ash like substance can be easily handled, transported, and used a fertilizer. The nuisance odors of transporting and treating organic waste are reduced by using this process. Thus, odor is reduced, clean water can be generated, and a marketable fertilizer results.

This disclosure has been described as having exemplary processes and is intended to cover any variations, uses, or adaptations using its general principles. It is envisioned that those skilled in the art may devise various modifications and equivalents without departing from the spirit and scope of the disclosure as recited in the following claims. Further, this disclosure is intended to cover such departures from the present disclosure as come within the known or customary practice within the art to which it pertains.

What is claimed is:

1. A vertical manure converter to accelerate processing of organic material using an activated carbon/organic material mixture, comprising:
a chamber with sections for controlling descent of the activated carbon/organic material mixture;
a plurality of air injectors along the chamber for inducing air into the chamber;
a heater in association with the chamber;
a means for reusing activated carbon; and
a separator suitable for separating ash.

2. The vertical manure converter of claim 1 wherein the air injectors induce compressed air at multiple levels along the chamber.

3. The vertical manure converter of claim 1 wherein the means for reusing activated carbon includes a screw auger to lift activated carbon inside the chamber from a lower portion of the chamber and a slinger connected to the screw auger adjacent to an entry area for organic material into the chamber.

4. The vertical manure converter of claim 1 wherein the means for reusing activated carbon includes a mixing reservoir for organic material and recycled or reactivated carbon from the chamber.

5. The vertical manure converter of claim 1 wherein an operating temperature is in a range of 300-450 degrees C.

6. The vertical manure converter of claim 5 wherein the operating temperature is in a range of 375-450 degrees C.

7. The vertical manure converter of claim 1 further comprising a dispersal cone in the chamber.

8. The vertical manure converter of claim 1 further comprising an outlet for exhaust stream from the chamber and a container with an activated carbon bed through which the exhaust stream passes to filter the exhaust stream.

9. The vertical manure converter of claim 8 further comprising an exhaust blower mounted on the container to help advance the exhaust stream into the activated carbon bed and a filtered exhaust port on the container above the activated carbon bed to further ensure clean final exhaust out of the container.

10. The vertical manure converter of claim 1 further comprising a computer system and temperature probes with relays to the computer system to control the amount of air injected into the chamber via the air injectors.

11. A vertical manure converter to accelerate composting process using an activated carbon/organic material mixture, comprising:
a chamber with internal sections for controlling descent of the activated carbon/organic material mixture;
a plurality of air injectors for inducing compressed air into the chamber;
a heater in association with the chamber;
a computer system;
a plurality of temperature probes with relays to the computer system for controlling the amount of air induced into the chamber via the air injectors; and
a separator suitable for separating ash.

12. A method of treating organic waste including the steps of:
mixing activated carbon into organic waste to form an activated carbon/organic waste mixture;
controlling descent of the activated carbon/organic waste mixture in a vertical chamber;
aerating the activated carbon/organic waste mixture in the vertical chamber;
reusing activated carbon; and
separating ash.

13. The method of claim 12 wherein the step of aerating the activated carbon/organic waste mixture includes injecting compressed air from air injectors along the chamber into descending activated carbon/organic waste mixture in the chamber.

14. The method of claim 12 wherein the step of reusing activated carbon includes collecting and transporting hot activated carbon to be mixed with organic waste in a continuing process of treating organic waste.

15. The method of claim 14 when the step of reusing activated carbon includes lifting activated carbon from a base of the chamber and slinging activated carbon at a top of the chamber wherein the activated carbon remains within the chamber.

16. The method of claim 12 including an additional step of monitoring and then regulating temperature in the vertical chamber by increasing or decreasing compressed airflow into the vertical chamber.

17. The method of claim 12 wherein the step of separating ash includes allowing ash to fall from a separation screen onto a sloped floor inside the chamber wherein the sloped floor gravity-feeds the ash to a discharge tube and using air pressure from inside the cylinder to facilitate feeding of ash to the discharge tube.

18. The method of claim 12 including an additional step of filtering exhaust steam by passing the exhaust steam through an activated carbon bed in a separate container.

19. The method of claim 12 including an additional step of providing steam from the method of treating organic waste.

20. The method of claim 12 including an additional step of heating the activated carbon/organic waste mixture to a temperature range of 375-450 degrees C.
